(19) United States
(12) Patent Application Publication
(10) Pub. No.: US 2013/0261470 A1
(43) Pub. Date: Oct. 3, 2013

(54) SYSTEMS AND METHODS FOR ESTIMATING BODY COMPOSITION

(71) Applicants: David Allison, Birmingham, AL (US); Olivia Thomas, Hoover, AL (US); Chengcui Zhang, Birmingham, AL (US)

(72) Inventors: David Allison, Birmingham, AL (US); Olivia Thomas, Hoover, AL (US); Chengcui Zhang, Birmingham, AL (US)

(21) Appl. No.: 13/992,070

(22) PCT Filed: Dec. 9, 2011
(86) PCT No.: PCT/US11/64220

§ 371 Date: Jun. 6, 2013

Related U.S. Application Data
(60) Provisional application No. 61/421,327, filed on Dec. 9, 2010.

(51) Int. Cl. A61B 5/107; A61B 5/00

(52) U.S. Cl. CPC A61B 5/1079; A61B 5/0077; A61B 5/7278; A61B 5/1073; A61B 5/4872; A61B 5/1077; A61B 5/7267
USPC 600/476

(57) ABSTRACT

In one embodiment, a system and method for estimating body composition relate to constructing a three-dimensional model of a subject based upon captured images of the subject, estimating the body volume of the subject using the three-dimensional model, and estimating the body composition of the subject based in part upon the estimated volume.

### CROSS-REFERENCE TO RELATED APPLICATION(S)

This application claims priority to co-pending U.S. Provisional Application Ser. No. 61/421,327, filed Dec. 9, 2010, which is hereby incorporated by reference herein in its entirety.

### BACKGROUND

Assessment of body composition, particularly fat and fat-free mass, is vital to understanding many health-related conditions, including cachexia induced by HIV, cancer, and other diseases; multiple sclerosis; wasting in neurological disorders such as Parkinson's, Alzheimer's, and muscular dystrophy; sarcopenia; obesity; eating disorders; proper growth in children; response to exercise; and yet others still. Nevertheless, challenges remain in the determination of these aspects of body composition.

Obesity, characterized by an excess amount of body fat, remains a significant public health problem. At the same time, sarcopenia is also becoming a major problem as our population ages. Sarcopenia refers to the diminution of lean body mass (primarily skeletal muscle) that accompanies aging and can lead to frailty and other health problems. Both obesity and sarcopenia can be assessed using sophisticated techniques such as dual-energy x-ray absorptiometry (DXA) or magnetic resonance imaging (MRI). Such methods are highly accurate and are often used in laboratory studies and in some clinical contexts. However, the methods are not widely used in large-scale epidemiologic studies and some field studies because of the cost, the difficulty in making these measurements portable, and the time it takes to do one measurement on one person, which is prohibitive in very large epidemiologic studies. Although calculation of body mass index (BMI) is a simpler method for estimating body composition, BMI is limited in value because it is an assessment of body weight relative to height and not of body composition per se.

Body fat estimation methods such as bioelectrical impedance analysis (BIA) are more portable and less expensive than DXA and can be used to measure body fat on large numbers of participants but are still limited in accuracy and require specialized equipment and time to implement.

From the above discussion, it can be appreciated that it would be desirable to have a means to inexpensively and accurately assess body composition without causing discomfort to the participant and without radiation exposure.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a schematic diagram of an embodiment of a system for estimating body composition.

FIG. 2 is a block diagram of an example configuration for an image capture device shown in FIG. 1.

FIG. 3 is a block diagram of an example configuration for a computer shown in FIG. 1.

FIG. 4 is a flow diagram of an embodiment of a method for estimating body composition.

FIG. 5 is a flow diagram of a further embodiment of a method for estimating body composition.

FIG. 6 is a diagram that illustrates generation of a three-dimensional model of a subject based upon two-dimensional images of the subject.

### DETAILED DESCRIPTION

As described above, it would be desirable to have a means to inexpensively and accurately assess body composition without causing discomfort to the participant and without radiation exposure. Disclosed herein are systems and methods for estimating body composition that satisfy those goals. In one embodiment, a system includes one or more image analysis algorithms that can be used to estimate the percent body fat of a subject from two-dimensional images of the subject. In some embodiments, the one or more image analysis algorithms can be executed on a portable device, such as a handheld device, that also is used to capture the images of the subject.

In the following disclosure, various embodiments are described. It is to be understood that those embodiments are example implementations of the disclosed inventions and that alternative embodiments are possible. All such embodiments are intended to fall within the scope of this disclosure.

Assessment of body composition, particularly fat mass (FM) and fat-free mass (FFM), is essential to the study of obesity and sarcopenia. In monitoring these diseases for response to treatment, monitoring the growth and loss of FM and FFM is fundamental. These are the most obvious and prevalent conditions for which measuring body composition is germane, yet many other conditions exist in which alterations in body composition abound and have important health impacts. For example, anorexia nervosa is characterized by a reduction of body mass to abnormal levels even after re-feeding and weight gain patients with anorexia nervosa have been shown to have reductions in FFM. Similarly, not only is Alzheimer's disease characterized by loss of weight and FFM, but such reductions appear to occur before and to presage the onset of cognitive deficits. So too are many other diseases associated with alternations in body composition including cachexia associated with cancer, HIV, neurologic disorders, congestive heart failure, and end-stage renal disease.

In such conditions of sarcopenia and wasting, and in response to exercise and other desired anabolic agents (e.g. exogenous hormone therapy), monitoring accretion of FFM is vital. In patients taking anti-psychotic, anti-retroviral, and some other pharmaceuticals, there are abnormalities in total weight, fat, and fat distribution. In settings where childhood malnutrition is a concern, monitoring proper growth requires the ability to monitor body composition. Recognizing the vital importance of body composition in these situations, investigators have for decades sought useful assessment methods. Although methods do exist, each has one or more drawbacks or limitations. Therefore, there is a vast unmet opportunity to improve translational science by offering an improved body composition assessment method.

Disclosed herein are systems and methods that are used to process digital photographic images of subjects (e.g., patients) and provide estimates of body fat percentage. Conceptually, the systems and methods build on two ideas. The first idea relates to Archimedes' Principle, which forms the basis for hydrodensitometry (UWW) and air displacement plethysmography (BodPod). In brief, if one knows the density of fat mass appendicular skeletal mass, and if the density of the whole body is known, one can determine the density of the whole body mass. The density can be calculated if both the mass and volume of the subject are known. Weight is usually determined by a conventional scale. Volume can be determined by the displacement of air, as in the BodPod, or, in the case of this disclosure, by using the visual information available in photographic images. Thus, the volume of a subject can be estimated and the density, and body composition, can be calculated therefrom.

The second idea builds on the observation that highly experienced and trained observers (e.g., body composition technologists) can estimate a person's body fat with reasonable accuracy by just looking at the person. For example, in the largest study to date, it was determined that visual estimates of percent body fat were moderately correlated with UWW estimates (r=0.78 males and r=0.72 females) in a sample of 1,069 military personnel. This observation indicates that there is sufficient information available in visual images to provide reasonable estimates of body composition. Such information may not be limited to simple estimates of volume. Indeed, common experience indicates that features such as “double chins,” jowls, the degree of sagging of flesh, the observability of lines of musculature, and other anatomical features all give clues to the individual's adiposity. A computer program or algorithm can be configured to detect these features, as well as others that humans may not be able to articulate, and use them to more accurately predict body composition. This may be referred to as the empirical-agnostic approach because it is based upon raw data crunching rather a priori identification of volume known to have theoretical relevance.

Before any analysis is performed, photographs of the subject must be captured. Perspective distortion is common in photographs and distorts the shape of the photographed subject. Specifically, the distortion makes the subject appear larger when the subject is close to the lens and or smaller when the participant is far from the lens. This phenomenon can introduce bias in the estimation of the size of the subject from photographs. Because, as described below, the accuracy of body volume estimation determines the accuracy of body-fat prediction, it may be necessary to correct perspective distortion as a post-digital processing step.

Two approaches can be effectively applied to reduce the impact of perspective distortion. The first approach focuses on correction with mathematical models by using a reference grid that provides standardized parallel lines. As the photographs are being captured, the subject can be positioned close to the reference grid marked on a background. After the photograph is captured, the reference grid can be used to correct the size as well as the orientation of the subject through a transformation process. In the second approach, the distance between the camera and the subject is increased to reduce the distortion. This approach is easy to apply but has the cost of losing certain image details. In some embodiments, the two approaches can be combined.

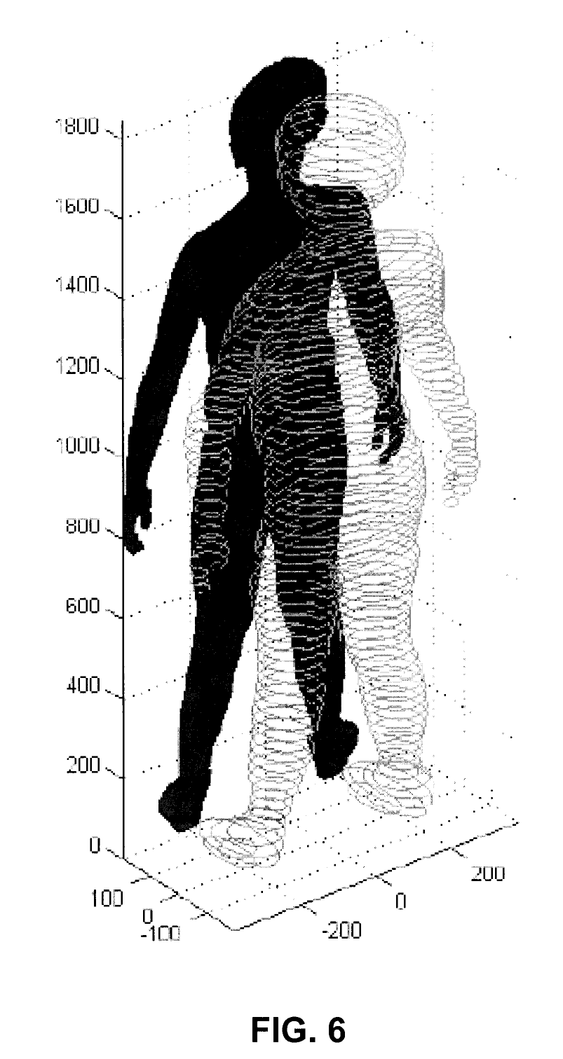

Digital images of the subject can be segmented to extract the two-dimensional (2D) object of the subject from each 2D image. A three-dimensional (3D) image synthesis algorithm can then be used to estimate the body volume of the subject. In some embodiments, horizontal ellipses are used to approximate cross-sections of the subject and estimate the body volume by accumulating the ellipses. The ellipse size can be determined by the major and minor semi-axes, which can be obtained from either the front-view or back-view image plus the side-view image. FIG. 6 illustrates a 3D model constructed from corresponding back and side profiles of a subject extracted from 2D images of the subject.

In some cases, ellipses may not accurately reflect the contours of a cross-dissection of the subject's body. Therefore, two alternative methods can be used to improve the approximation accuracy. The first alternative involves replacing the ellipse with a more refined contour based upon existing knowledge about the shape of cross-dissections of different human body parts learned from computed tomography (CT) scans. If the contours of a cross-section are estimated with a contour template obtained from a real person, more accurate results are likely, as compared to methods in which ellipses are used. In some cases, an arbitrary CT scan serve as the contour template and can be rescaled according to the width, depth, and height information obtained from the images.

The second alternative volume estimation method is motivated by the monophotogrammetry approach proposed by Pierson in 1961. This method uses a single camera, two flashing units, and a two-sided color filtering system to capture two images of the subject from the front and the back, respectively. Body volume can be estimated based on the 2D area information on manually traced color isopleths and the known width of the color strips. As a further alternative a single camera and a single color light source can be used from the front instead of projecting lights through color strips from both sides. By applying digital image processing techniques, the light intensity reflected by the human body surface can be easily and relatively reliably extracted from front/back view photographs. This method reduces the imprecision due to the depth discretization using color strips and there is no complex calibration process involved.

Once a 3D volume model of the subject has been constructed, visual cues such as body shape, the size of the neck, hips, and waist, and facial characteristics can be extracted. In some cases, these visual cues can be identified after segmenting the 3D model into four parts: head, neck, torso, and limbs. During the segmentation process, 3D morphological analysis can be performed to divide the body into different parts during the segmentation process. The visual cues can be considered to be additional clues indicating the level of fat mass and appendicular skeletal muscle, and therefore be used to fine tune the body composition estimate.

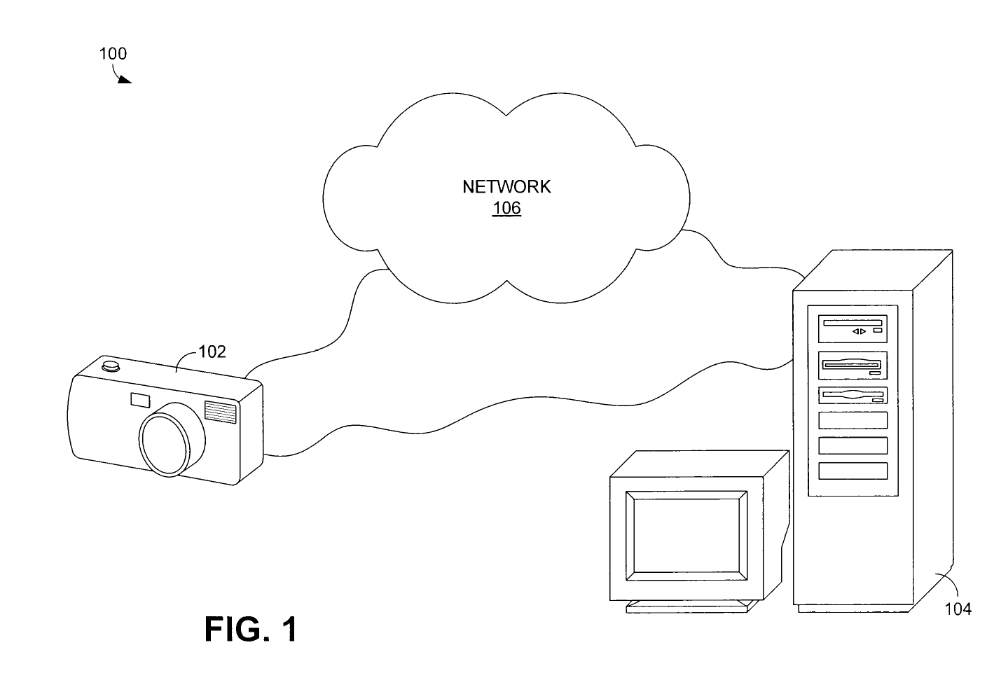

FIG. 1 illustrates an example system 100 for estimating body composition. As indicated in the figure, the system 100 comprises a portable (e.g., handheld) image capture device 102 and a computer 104 to which image data captured with the image capture device can be transmitted for analysis. By way of example, the image capture device 102 comprises a digital camera. Alternatively, however, the image capture device 102 can be another device that is adapted to capture images but that may have other functionality also. For example, the image capture device could comprise a mobile phone (e.g., a “smart phone”) or a tablet computer. Therefore, in some embodiments, the image capture device can be considered to be a computing device. As is also indicated in FIG. 1, the computer 104 can comprise a desktop computer. Although a desktop computer is shown in FIG. 1, the computer 104 can comprise substantially any computing device that can receive image data from the image capture device 102 and analyze that data. Accordingly, the computer 104 could comprise, for example, a notebook computer or a tablet computer.

The image capture device 102 can communicate with the computer 104 in various ways. For instance, the image capture device 102 can directly connect to the computer 104 using a cable (e.g., a universal serial bus (USB) cable) that can be plugged into the computer 104. Alternatively, the image capture device 102 can indirectly “connect” to the computer 104 via a network 106. The image capture device's connection to such a network 106 may be via a cable (e.g., USB cable) or, in some cases, via wireless communication.

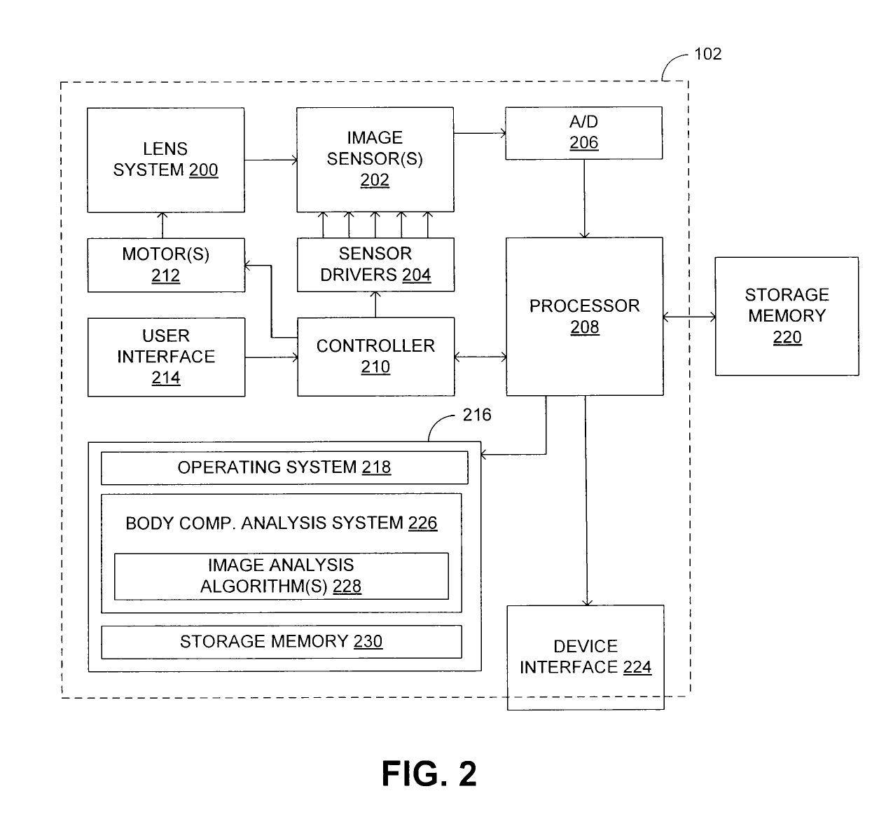

FIG. 2 illustrates an example configuration for the image capture device 102 shown in FIG. 1. The image capture device 102 includes a lens system 200 that conveys images of viewed scenes to an image sensor 202. By way of example, the image sensor 202 comprises a charge-coupled device (CCD) or a complementary metal oxide semiconductor (CMOS) sensor that is driven by one or more sensor drivers 204. The analog image signals captured by the sensor 202 are provided to an analog-to-digital (A/D) converter 206 for conversion into binary code that can be processed by a processor 208. Such components can be generally referred to as image capturing apparatus.

Operation of the sensor driver(s) 204 is controlled through a device controller 210 that is in bi-directional communication with the processor 208. The controller 210 also controls one or more motors 212 (if present) that can be to drive the lens system 200 (e.g., to adjust focus and zoom). Operation of the device controller 210 may be adjusted through manipulation of a user interface 214. The user interface 214 comprises the various components used to enter selections and commands into the image capture device 102 and therefore can include various buttons as well as a menu system that, for example, is displayed to the user in a display of the image capture device (not shown).

The digital image signals are processed in accordance with instructions from an operating system 218 stored in permanent (non-volatile) device memory 216. Processed (e.g., compressed) images may then be stored in local storage memory 230 or an independent storage memory 220, such a removable solid-state memory card (e.g., Flash memory card).

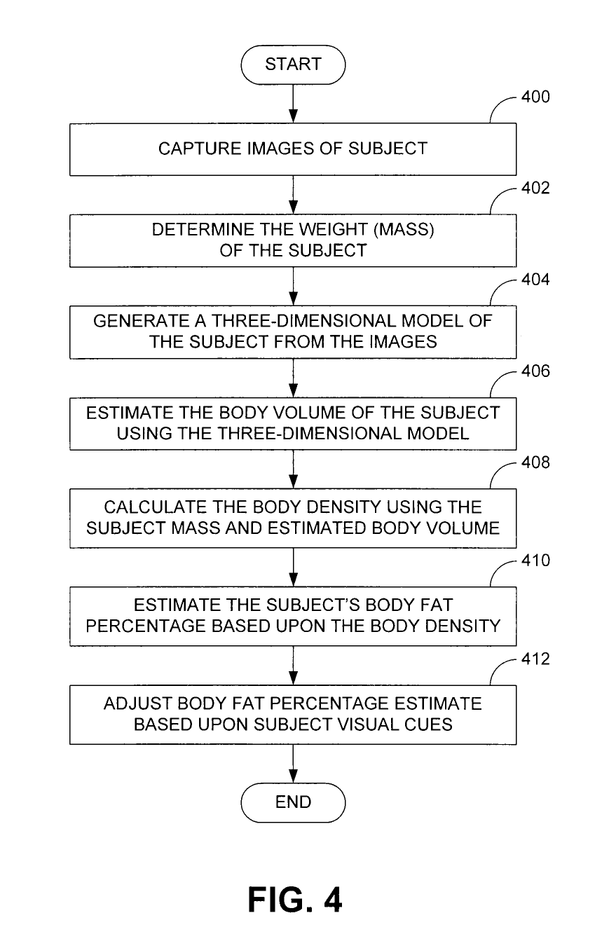

In the embodiment of FIG. 2, the device memory 216 further comprises a body composition analysis system 226 that includes one or more image analysis algorithms 228 that are configured to analyze images of subjects for the purpose of estimating their body compositions from the images. Examples of this process are described below in relation to FIGS. 4-6. Notably, the body composition analysis system 226 could alternatively be hard coded into a separate chip provided within the image capture device 102.

The image capture device 102 further includes a device interface 224, such as a universal serial bus (USB) connector, that is used to connect the image capture device 102 to another device, such as the computer 104.

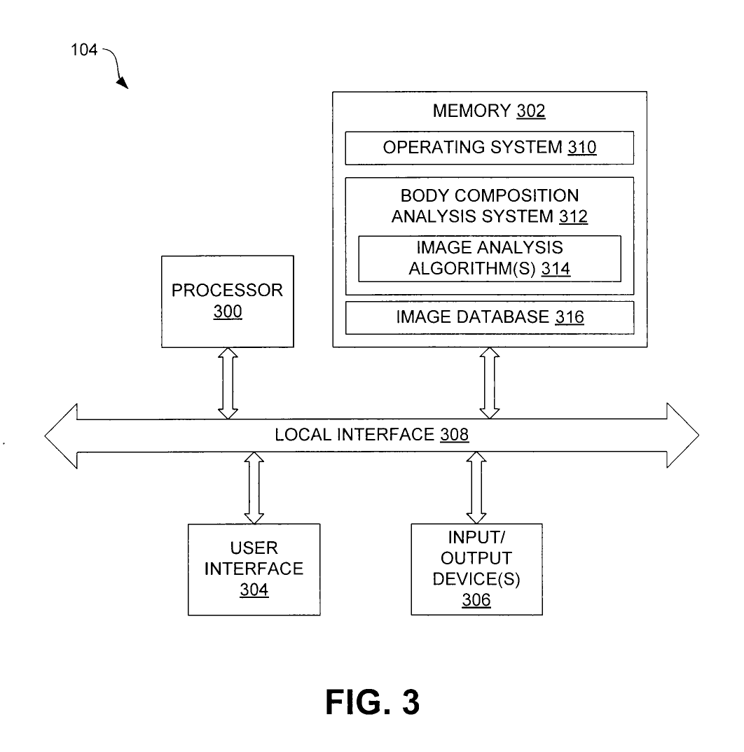

FIG. 3 illustrates an example configuration for the computer 104 shown in FIG. 1. As is indicated in FIG. 3, the computer 104 comprises a processor 300, memory 302, a user interface 304, and at least one input/output (I/O) device 306, each of which is connected to a local interface 308.

The processor 300 can comprise a central processing unit (CPU) or other processor. The memory 302 includes any one of or a combination of volatile memory elements (e.g., RAM) and nonvolatile memory elements (e.g., read only memory (ROM), Flash memory, hard disk, etc.).

The user interface 304 comprises the components with which a user interacts with the computer 104, such as a keyboard and mouse, and a device that provides visual information to the user, such as a liquid crystal display (LCD) monitor.

With further reference to FIG. 3, the one or more I/O devices 306 are configured to facilitate communications with the image capture device 102 and may include one or more communication components such as a modulator/demodulator (e.g., modem), USB connector, wireless (e.g., (RF)) transceiver, or a network card.

The memory 302 comprises various programs including an operating system 310, a body composition analysis system 312 that includes one or more image analysis algorithms 314, each of which can function in similar manner to the like-named elements described above in relation to FIG. 2. In addition, the memory 302 comprises an image database 316 in which images received from the image capture device 102 can be stored.

Various programs have been described above. These programs comprise computer instructions (logic) that can be stored on any non-transitory computer-readable medium for use by or in connection with any computer-related system or method. In the context of this disclosure, a computer-readable medium is an electronic, magnetic, optical, or other physical device or means that contains or stores a computer program for use by or in connection with a computer-related system or method. These programs can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

FIG. 4 is a flow diagram that describes a method for estimating body composition that is consistent with the disclosure provided above. In the flow diagrams of this disclosure, various actions or method steps are described. It is noted that the actions/steps can, in some cases, be performed in an order other than that implied by the flow diagrams. Moreover, in some cases actions/steps can be performed simultaneously.

Beginning with block 400 of FIG. 4, digital images of a subject whose body composition is to be estimated are captured. As described above, the images can be captured using a digital camera or another device that is capable of capturing digital images. In some embodiments, the images can be captured using a dedicated device specifically intended for use in body composition estimation that can capture and process the image, as well as provide a body composition estimate.

In some embodiments, images are captured from multiple sides of the subject. For example, front-view, side-view (profile), and rear-view images can be captured of the subject. Notably, however, a front view and a side view pair, or a rear view and a side view pair, may be sufficient to perform the body composition estimation.

Referring next to block 402, the weight (as well as mass), of the subject is determined. By way of example, this simply comprises weighing the subject on a scale. As described below, the subject's mass is useful in estimating the density of the subject, which can then be used to calculate the subject's body fat percentage.

Turning next to block 404, a 3D model of the subject is generated from the captured images. Although it is possible to generate the 3D model manually, it may be preferable to use an image analysis algorithm, such as algorithm 228 (FIG. 2) or algorithm 314 (FIG. 3), to automatically generate the 3D model from the images.

After the 3D model of the subject has been generated, the subject's body volume can be estimated using the model, as indicated in block 406. As described below, this process can be automated by a body composition analysis system, such as the system 226 (FIG. 2) or the system 312 (FIG. 3). In some embodiments, the system can estimate the volume by dividing the 3D model into elliptical segments that emulate the volumes of discrete portions of the model (and therefore the subject), and then adding the discrete volumes together to obtain a total volume. This process is pictorially illustrated in FIG. 6.

Once the subject's mass and volume are known, the subject's body density can be calculated (block 408) by dividing the mass by the volume. Once the subject's density is known, the subject's body fat percentage can be estimated (block 410) using the following equation:

PBF=(495/BD)−450 Equation 1

where PBF is percent body fat and BD is body density.

It is noted that the subject's body fat percentage can be calculated in other ways using the body volume. For example, the fat mass can be calculated from the body volume and body weight, and the fat mass can then be used to calculate body fat percentage using the following equations:

FM=4.95(BV)−4.5(BW) Equation 2

PBF=100(FM/TBM) Equation 3

where FM is fat mass, BV is body volume, BW is body weight, and TBM is total body mass.

Through the above-described process, a good estimate of the subject's body fat percentage is obtained. In some embodiments, the accuracy of the estimate can be increased by considering various visual cues. As described above, such visual cues can include body shape, the size of the neck, hips, and waist, and facial characteristics. Other cues may comprise jowls, “love handles,” pot bellies, skin rolls, and any other body feature that is indicative of the amount of body fat that the subject carries. Therefore, the body fat percentage estimate can be adjusted based upon the visual cues, as indicated in block 412. In some embodiments, the image analysis algorithm can automatically identify the visual cues and the body composition analysis system can adjust the body fat percentage estimate in view of those cues.

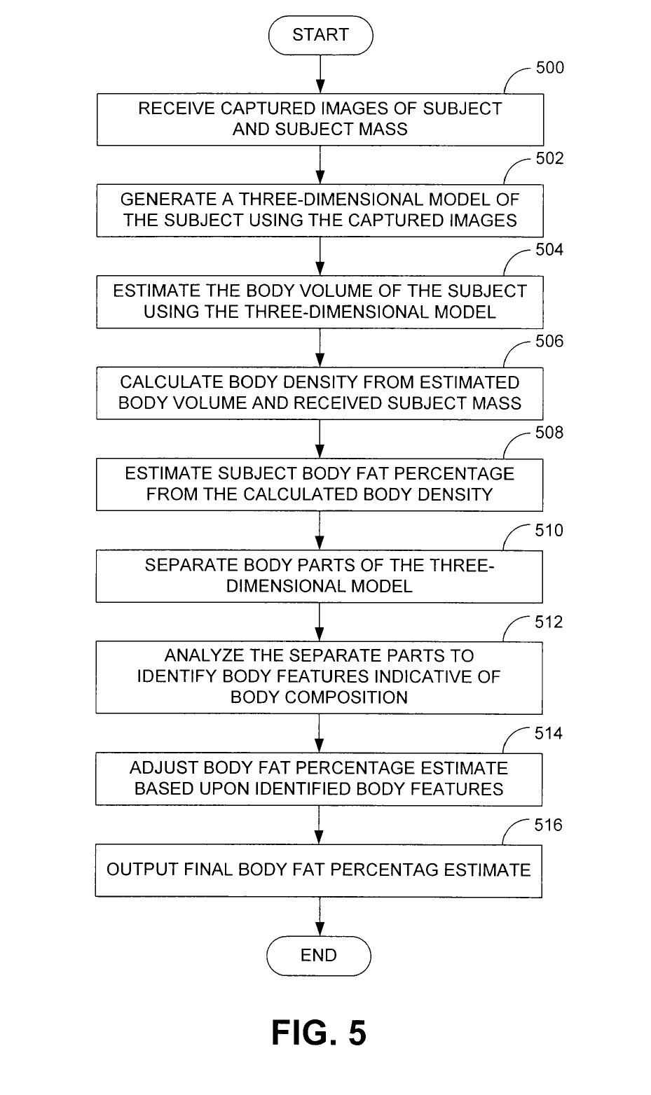

FIG. 5 is a flow diagram that describes a further method for estimating body composition. More particularly, FIG. 5 describes a method for estimating body composition using a computing device, which can be an image capture device or a computer. For purposes of discussion, the term “computing device” will be used to refer to the device (camera, computer, or otherwise) that performs the method described in FIG. 5.

Beginning with block 500, the computing device receives captured images of the subject and the subject's mass. As noted above, the images can comprise images captured by an image capture device (either the computing device itself or another device capable of capturing digital images). The subject's mass can have been manually input into the computing device using an appropriate user interface.

Once the images have been received, the computing device generates a 3D model of the subject using the images, as indicated in block 502. The computing device can then estimate the body volume of the subject using the 3D model, as indicated in block 504. As noted above, the volume can be estimated by segmenting the 3D model into discrete elliptical portions that estimate the shape of the various parts of the model (and therefore the subject's body), determining the volume of each discrete portion, and adding the discrete volumes together to obtain a total body volume. Alternatively, contours of a cross-section of a contour template can be used instead of ellipses.

With the body mass and body volume, the computing device can calculate the body density (block 506) and estimate the body fat percentage (block 508), for example using Equation 1.

At this point, the computing device can refine the body fat percentage estimate by considering various physical attributes of the subject's body, as represented by the 3D model. In some embodiments, this process involves separating the model into separate body parts (block 510) and analyzing the separate parts to identify body features that are indicative of the subject's body composition (block 512). As noted above, such features can be double chins, jowls, love handles, pot bellies, etc. The algorithm used to estimate body composition can take one or more of these visual cues into account and adjust the body fat estimate to increase its accuracy (block 514). For example, if the image analysis reveals that the subject has a protruding belly and love handles, the algorithm may increase the body fat percentage estimate given that such physical attributes tend to appear in subjects that have higher body fat percentages.

Once the body fat percentage estimate has been adjusted, if such adjustment was necessary, the computing device outputs a final body fat percentage estimate to the user (e.g., medical professional), as indicated in block 516.

Claimed are:

1. A method for estimating body composition of a subject, the method comprising:
capturing images of the subject;
constructing a three-dimensional model of the subject based upon the images;
estimating the body volume of the subject using the three-dimensional model; and
estimating the body composition of the subject based in part upon the estimated volume.

2. The method of claim 1, wherein capturing images comprises capturing digital images of the subject.

3. The method of claim 1, wherein capturing images comprises capturing a profile image and at least one of a front image or a back image of the subject.

4. The method of claim 1, wherein estimating the body volume of the subject comprises dividing the three-dimensional model into discrete elliptical segments, calculating the volume of each elliptical segment, and summing the volumes of all elliptical segments to obtain a total volume.

5. The method of claim 1, wherein estimating the body volume of the subject comprises dividing the three-dimensional model into discrete segments whose shape is based upon the contours of an actual cross-dissection of a human body, calculating the volume of each segment, and summing the volumes of all segments to obtain a total volume.

6. The method of claim 1, wherein estimating body composition of the subject comprises estimating body density of the subject from the estimated body volume and the mass of the subject.

7. The method of claim 6, wherein estimating body composition of the subject further comprises calculating the subject's body fat percentage using a relation that directly relates body fat percentage to body density.

8. The method of claim 1, wherein estimating body composition of the subject comprises estimating fat mass of the subject from the estimated body volume and the weight of the subject, and then calculating the subject's body fat percentage using relation that directly relates body fat percentage to fat mass and total mass of the subject.

9. The method of claim 1, further comprising analyzing the images to identify visual cues indicative of the subject's body composition.

10. The method of claim 9, further comprising adjusting the body composition estimate based upon the visual cues.

11. A system for estimating body composition of a subject, the system comprising:
a processor; and
memory that stores a body composition analysis system, the system being configured to receive images of a subject, to construct a three-dimensional model of the subject based upon the images, to estimate the body volume of the subject using the three-dimensional model, and to estimate the body composition of the subject based in part upon the estimated volume.

12. The system of claim 11, wherein the system is embodied by an image capture device that further comprises image capturing apparatus.

13. The system of claim 11, wherein the system is embodied by a computer.

14. The system of claim 11, wherein the body composition analysis system is configured to estimate the body volume of the subject by dividing the three-dimensional model into discrete elliptical segments, calculating the volume of each elliptical segment, and summing the volumes of all elliptical segments to obtain a total volume.

15. The system of claim 11, wherein the body composition analysis system is configured to estimate the body volume of the subject by dividing the three-dimensional model into discrete segments whose shape is based upon the contours of an actual cross-dissection of a human body, calculating the volume of each segment, and summing the volumes of all segments to obtain a total volume.

16. The system of claim 11, wherein the body composition analysis system is configured to estimate body composition of the subject by estimating body density of the subject from the estimated body volume and the mass of the subject.

17. The system of claim 16, wherein the body composition analysis system if further configured to estimate body composition of the subject by calculating the subject's body fat percentage using a relation that directly relates body fat percentage to body density.

18. The system of claim 11, wherein the body composition analysis system is configured to estimate body composition of the subject by estimating fat mass of the subject from the estimated body volume and the weight of the subject, and then calculating the subject's body fat percentage using relation that directly relates body fat percentage to fat mass and total mass of the subject.

20. The system of claim 11, wherein the body composition analysis system is further configured to analyze the images to identify visual cues indicative of the subject's body composition.

21. The system of claim 20, wherein the body composition analysis system is further configured to adjust the body composition estimate based upon the visual cues.

22. An image capture device, comprising:
image capturing apparatus;
a processor; and
memory that stores a body composition analysis system, the system being configured to receive images of a subject, to construct a three-dimensional model of the subject based upon the images, to estimate the body volume of the subject using the three-dimensional model, and to estimate the body composition of the subject based upon the estimated volume.
